(19) United States
(12) Patent Application Publication
(10) Pub. No.: US 2006/0252050 A1
(43) Pub. Date: Nov. 9, 2006

(54) Genetic marker for weight regulation

(76) Inventors: Jose M. Ordovas, Framingham, MA (US); Dolores Corella, Valencia (ES)

Correspondence Address: DAVID S. RESNICK, 100 SUMMER STREET, NIXON PEABODY LLP, BOSTON, MA, 02110-2131, US

(21) Appl. No.: 11/123,845

(22) Filed: May 6, 2005

(51) Int. Cl. C12Q 1/68

(52) U.S. Cl. 435/6

(57) ABSTRACT

The present invention is directed to methods capable of predicting likely response to weight loss and weight management based on genetic polymorphisms in the perilipin (PLIN) locus. The invention also provides kits to determine whether an individual is resistant to weight gain or weight loss based on analysis of genetic polymorphisms at the perilipin locus. This information can be used to screen individuals, such as obese and overweight individuals and classify them based on their genetic tendency to either lose weight or resistance to lose weight. Similarly, the polymorphisms can be used to identify individuals who are underweight, such as anorectic individuals, who could be genetically resistant to weight-gain using dietary intervention alone. Screening of normal weight individuals could help to identify people who are resistant to gaining or losing weight, or alternatively individuals, who are more susceptible for weight changes either to extreme high or low. Appropriate measures can then be implemented in life-style, diet, medicinal and possible surgical interventions. Such a genetic approach will help professionals in the field of weight-management to improve targeting patients with appropriate advise regarding their weight management.

### GOVERNMENT SUPPORT

This invention was supported by the National Institutes of Health under grant No. HL54776, and U.S. Department of Agriculture Research Service under contracts No. 53-K06-5-10 and No. 58-1950-9-001. Therefore, the U.S. Government has certain rights to the invention.

### BACKGROUND

1. Field of the Invention

The present invention is related to genetic tests and methods. Particularly, the invention is directed to methods to assess an individual's likelihood of responsiveness to weight management program by genetically classifying individuals as likely susceptible or likely resistant to weight management programs, for example, weight management programs comprising a dietary intervention.

2. Background of the Invention

It is clear that not all individuals can lose weight with one standard protocol. This is cause for a great debate in discussions of how to alleviate the currently globally spreading obesity epidemic. Obesity is a serious medical condition that currently affects about one third of adults in the United States, and about 14% of children and adolescents. In Europe, the number of obese children is currently estimated to rise by about 400,000 children a year (International Obesity Taskforce EU Platform Briefing Paper, Mar. 15, 2005, at www.iotf.org). The abundance of energy sources and the sedentary lifestyle in developed countries has made obesity a worldwide phenomenon. In the United States, obesity can currently be said to be the second leading cause of preventable death after smoking (www.obesity.org).

Obesity is a complex condition with serious biological, psychological and social repercussions and threatens to overpower the health systems (1). While is the best way to attack the problem is prevention, there is also a great need for tools to assist professionals involved in weight-loss programs for the currently obese individuals and to help these individuals gain a healthier weight (2).

In addition to the current obesogenic environment, genetic factors play a role in the predisposition of individuals to developing obesity and on the efficacy of current therapies (3). Therefore, public health efforts and treatment strategies could be dramatically improved if predictive information about the response of the obese subject to intervention (i.e., energy restriction) was available.

Although the evidence strongly supports low energy diets as the optimal choice for weight loss, there is still controversy surrounding different dietary patterns (low-fat, low carbohydrates, etc.) used to promote weight loss, and none has emerged definitively as more effective (4-6). On the other hand, the increasing knowledge of the genes involved in the development of obesity is paving the way for new approaches of obesity control because it is unlikely that one diet is optimal for all obese persons. In this sense, nutritional genomics will provide the basis for personalized dietary recommendations based on the individual's genetic make up (7). This implies that some individuals are more susceptible to body-weight gain or loss than others because of genetic differences. However, before nutritional genomics is in a position to contribute significantly to treatment of obese patients, an enormous amount of work has to be done to identify relevant genetic variants their specific interactions (8).

During the evolution, the human body has developed a vast variety of ingenious ways to cope with lack of calorie intake to avoid starvation, and only recently have we began to realize the complexity of these metabolic networks. During the present times of abundance in calorie input in the developed world, this intricate and complex system has began to work against us resulting in severe epidemic of obesity and related metabolic diseases.

Studies relating to genetics of obesity classify obesity phenotypes using various parameters including, for example, body mass or body fatness, body fat distribution (abdominal visceral fat, waist circumference, waist-to-hip girth ratio, and sagittal diameter), resting energy expenditure, thermic effect of feeding, 24-hour lipid oxidation rate, adipocyte size, number and lipolysis rate, and plasma leptin levels. A number of genes have been shown to be associated with each of the above-listed phenotypes (see, e.g. Snyder et al., The Human Obesity Gene Map: The 2003 Update, Obesity Research 12(3): 369-439, 2004). Also, a polymorphism in GNB3 gene has been found to be associated with pregnancy-related weight gain. Loci associated with weight loss have also been identified. For example, polymorphisms in GNB3 and PNMT loci have been shown to be predictive of sibutratime-induced weight loss. A marker in ADRB2 locus has been shown to be associated with responsiveness to “lifestyle” based weight-loss program, and endurance training-induced changes in body composition have been shown to be associated with polymorphisms in ADRB2 gene (see, e.g. Snyder et al., The Human Obesity Gene Map: The 2003 Update, Obesity Research 12(3): 369-439, 2004).

Perilipins are proteins in adipocytes that functions to increase cellular triglyeride storage and mobilization. Perilipin knockout mice are lean and resistant to diet-induced obesity. In this regard, adipose tissue plays a central role in regulating energy storage and mobilization, and it has been the focus of efforts to identify candidate genes for obesity and weight management. Perilipins are phosphorylated proteins in adipocytes that are localized at the surface of the lipid droplet (9, 10). Experimental studies have shown that these proteins are essential in the regulation of triglycerides deposition and mobilization (11-13). After activation of protein kinase A, perilipin is phosphorylated, resulting in translocation of the protein away from the lipid droplet and allowing hormone-sensitive lipase to hydrolyze the adipocyte triglycerides to release nonesterified fatty acids (14, 15). Perilipin functions to increase cellular triglycerides storage by decreasing the rate of triglycerides hydrolysis and serves an additional role in controlling the release of triglycerides at times of need. Two independent laboratories produced perilipin knockout mice (16, 17), and shown that these animals were lean, had increased basal lipolysis, and were resistant to diet-induced obesity.

Despite all the gene association studies in this field and the mounting detailed knowledge of the individual proteins, there still exists very few useful markers to guide a physician advising an obese patient how best to reduce weight and keep the weight-loss permanent. Dietary treatment of obesity could be drastically improved if predictive information about the genetic response to diet was available.

### SUMMARY OF THE INVENTION

Accordingly, the present invention is directed to methods capable of predicting likely response to weight loss and weight management based on genetic polymorphisms in the perilipin (PLIN) locus. The invention also provides kits to determine whether an individual is resistant to weight gain or weight loss based on analysis of genetic polymorphisms at the perilipin locus. This information can be used to screen individuals, such as obese and overweight individuals and classify them based on their genetic tendency to either lose weight or resistance to lose weight. Similarly, the polymorphisms can be used to identify individuals who are underweight, such as anorectic individuals, who could be genetically resistant to weight-gain using dietary intervention alone. Screening of normal weight individuals could help to identify people who are resistant to gaining or losing weight, or alternatively individuals, who are more susceptible for weight changes either to extreme high or low. Appropriate measures can then be implemented in life-style, diet, medicinal and possible surgical interventions. Such a genetic approach will help professionals in the field of weight-management to improve targeting patients with appropriate advise regarding their weight management.

The invention is based on the finding that carriers of the 11482A allele in the PLIN4 locus (11482G>A) had great difficulty in losing weight with a reduced-calorie diet. Also, the same PLIN4 “A” allele carries, who are normal weight, are generally more resistant to either losing or gaining weight. Thus, this polymorphism is useful in predicting the outcome of body-weight management strategies, particularly having a component of dietary intervention, such as low-energy or low calorie, or alternatively high energy or high-calorie diets.

Accordingly, the invention provides a method of predicting an individual's response to a weight management program the method comprising analyzing the individual's genotype at the perilipin loci, wherein the presence of either one or two PLIN4 allele “A” is indicative of the individual being likely resistant to weight change, preferably resistant to weight change when weight management program comprises dietary intervention either alone or as its main component.

In one embodiment, the invention provides a method of determining whether an overweight or obese individual is a suitable candidate, i.e. susceptible for weight-loss program, or for weight-management program comprising a dietary component alone or as its main component, for example, low-energy diet also called low calorie diet. The method comprises genotyping the PLIN loci, preferably at least the PLIN4 locus, of the overweight or obese individual, wherein the absence of PLIN4 allele “A” is indicative of the individual being a good candidate for weight-management by a low-energy diet.

Alternatively, the invention provides a method of determining whether an individual in a normal weight range, is a suitable candidate, i.e. susceptible for weight-management by a program comprising a dietary component alone or as its main component, for example, low-energy diet also called low calorie diet. The method comprises genotyping the PLIN loci, preferably at least the PLIN4 locus, of the individual, wherein the absence of PLIN4 allele “A” is indicative of the individual being a good candidate for weight management program with a dietary component.

In one embodiment, the invention provides a method of determining whether an overweight or obese individual is not a suitable candidate, i.e. susceptible for weight loss, for weight-management program comprising a dietary component, such as low-energy or low calorie diet. The method comprises genotyping the PLIN loci, preferably at least the PLIN4 locus, of the overweight or obese individual, wherein the presence of one or two PLIN4 allele “A” is indicative of the individual not being a good candidate for weight-management by a low-energy diet alone.

In one embodiment, the invention provides a kit for determining whether an individual is an appropriate candidate to weight management program, preferably to a program that comprises a dietary intervention component, for example, low-energy diet, wherein the kit comprises genotyping means for PLIN loci, preferably at least PLIN4 locus or any other PLIN locus in tight linkage disequilibrium with PLIN4 locus, and an instruction manual explaining that detection of at least one allele A at PLIN4 locus in indicative of the individual as being not susceptible for weight management by dietary, particularly low-calorie, intervention, and that detection of other than A, allele, such as detection of an individual homozygous for the G allele at the PLIN4 locus, is indicative of that individual being susceptible, i.e. a good candidate to weight management using dietary, for example low-calorie intervention either alone or as one major component of the weight management program.

### BRIEF DESCRIPTION OF DRAWINGS

FIG. 1 shows mean body-weight in the 48 obese patients (9 men and 39 women) that participate in the dietary intervention study at baseline, 3 months, 6 months and 1 year of follow-up, depending on the PLIN11482 polymorphism. Results were adjusted for gender and age. P for the interaction term was obtained in the ANCOVA for repeated-measures in the model adjusted for gender and age.

FIG. 2 shows Table 1.

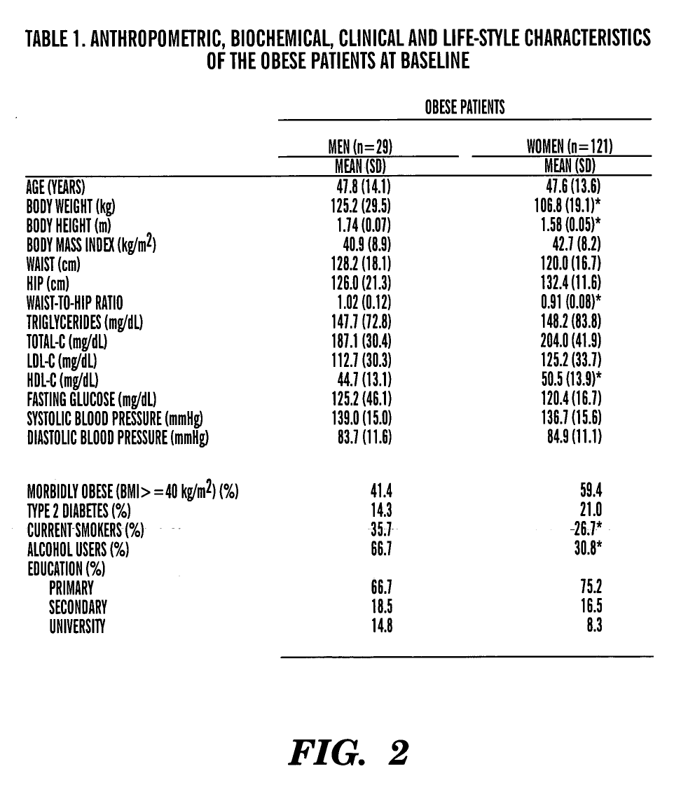

FIG. 3 shows Table 2.

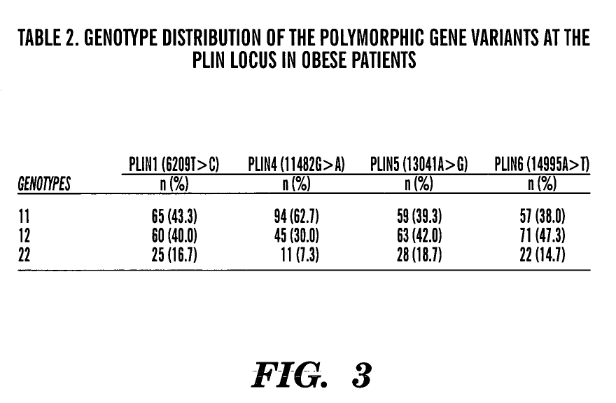

FIG. 4 shows Table 3.

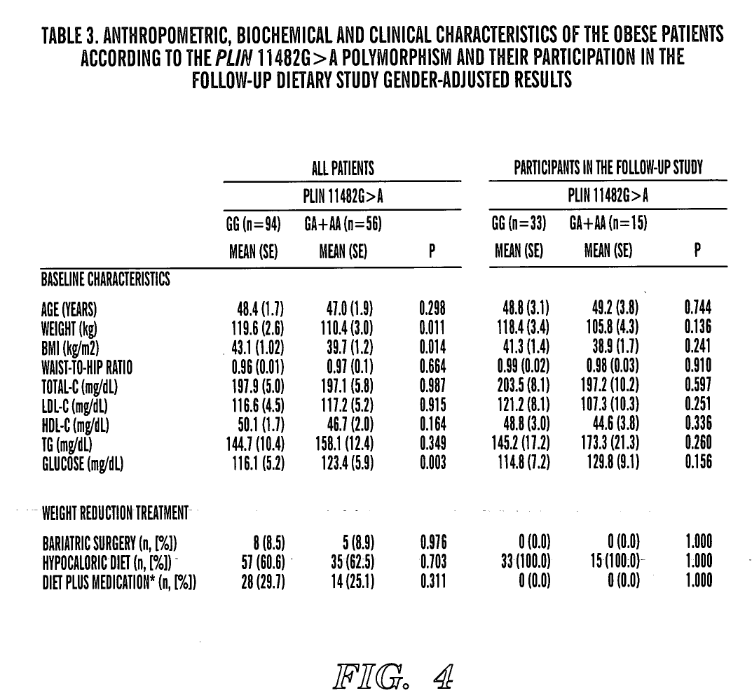

FIG. 5 shows Table 4.

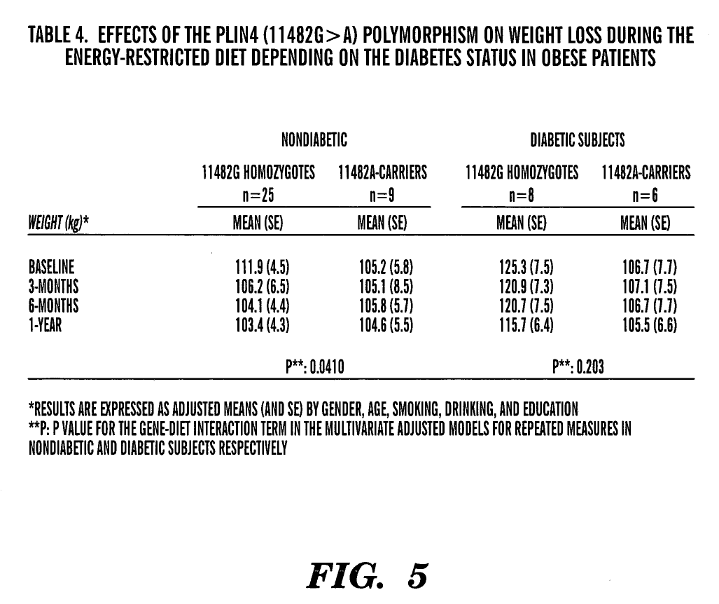

### DETAILED DESCRIPTION OF THE INVENTION

The present invention provides an allele of PLIN5 locus, allele A at PLIN11482, that is associated with resistance to weight modulation or management, particularly by diet, such as low-energy or low-calorie diets. Accordingly, the present invention also provides methods of determining whether an individual, preferably an overweight or obese individual, is susceptible, or a suitable candidate, for weight management using dietary intervention, preferably low-energy or low-calorie diet.

The invention is based on the finding that individuals carrying certain PLIN alleles were found to be more resistant to weight change, such as weight loss or weight gain, than individuals carrying other PLIN alleles. We examined the association between four polymorphisms at the perilipin (PLIN) locus (PLIN1: 6209T>C, PLIN4: 11482G>A, PLIN5: 13041A>G, and PLIN6: 14995A>T) with obesity and weight reduction in response to a low-energy diet in morbidity obese patients (body mass index mean, 42±8 kg/m2). The 11482G>A polymorphism was statistically significantly associated with weight in the obese patients at baseline (n=150). Moreover, we found a gene-diet interaction (P=0.015) between this polymorphism and the dietary intervention in determining body-weight in patients that completed the one-year dietary follow-up treatment consisting of a low-energy diet and mean body weight (from 114.3±3.9 kg at baseline to 105.5±3.5/kg at 1-year; P-lineal trend: 0.020) in patients with wild-type genotype (GG, n=33). Conversely, patients with the variant allele (A-carriers, n=15) did not show significant changes in mean body weight (from 105.0±4.6 kg at baseline to 104.3±4.4 kg at 1-year; P-lineal trend: 0.985). Also, individuals with normal weight and carrying the PLIN4 “A” allele, appear to be resistant to gaining weight.

These results indicate that carriers of the 11482A allele had a great difficulty in managing their weight, surprisingly showing that this polymorphism can predict outcome of, for example, body-weight reduction strategies that are based on dietary intervention, such as low-energy diets.

Consequently, the identification of the PLIN4 “A” allele carriers can help weight management professionals to design alternative weight management programs for these individuals. Alternatives to low-energy diets include increase in physical activity, behavior therapy, drug treatment, particularly drugs increasing energy consumption rather that limiting energy absorption, surgery, dietary supplements and liposuction. Individuals carrying PLIN4 allele “A” would likely benefit of a combination of one or more of the methods listed above either with or without a low-energy diet.

Alternatively, underweight individuals carrying one or two PLIN4 “A” alleles, may have difficulties in gaining weight using weight management programs having diet as a sole, or major component of the program. Consequently, the methods of identifying PLIN polymorphisms, particularly PLIN 4 genotype, could help to council the weight management in these individuals to include, for example, energy-consumption-reducing life-style, or pharmaceutical intervention.

The method of the present invention can also be used in screening individuals of the general population, such as teenagers, who may be overly conscious of their weight, even if it falls into the so called “normal” range, one definition of which is BMI 18.5-24.9. Identification of PLIN4 “A” allele in these individuals could provide health professionals with tools to discuss about the difficulties of an individual with a BMI of 24 to reach BMI of 22 with a lower-calorie diet alone.

Possible pharmaceutical interventions include, but are not limited to rimonabant, which blocks the same pleasure receptor in the brain that responds to marijuana (marketed under the name Acomplia by Sanofi-Aventis, SA,), intranasal PYY3-36 (PYY is a naturally occurring human hormone produced by specialized endocrine cells (L-cells) in the gut in proportion to the calorie content of a meal, PYY3-36 is a modified form of PYY and is studied by Nastech Pharmaceutical Company Inc.), Xenical, a molecule that attaches to lipases and blocks them from breaking down some of the fat in the diet (Roche), and sibutramine hydrochloric monohydrate which acts as a monoamine (serotonin and norepinephrine) re-uptake inhibitor and affects the feeling of satiety (marketed under name Meridia, made by Abbot Laboratories). Preferable pharmaceuticals include energy consumption-increasing drugs, β3-adrenergic receptor agonists, and PPARγ agonists.

The perilipin or PLIN locus as used herein refers to loci including, but not limited to PLIN1 at nucleotide 6252 of sequence with GenBank accession no. gi21431190, PLIN4 at nucleotide 11482 of sequence with GenBank accession no. gi21431190, PLIN5 at nucleotide 13041 of sequence with GenBank accession no. gi21431190, and PLIN6 at nucleotide 14995 of sequence with GenBank accession no. gi21431190.

The PLIN allele refers to alleles with at least one of the two possible nucleic acids at the PLIN locus, and comprise at least the following polymorphic markers or any markers that are in tight linkage disequilibrium with them: PLIN1: 6209T (major allele in general population)>C (minor allele in general population), PLIN4: 11482G (major allele in general population)>A (minor allele in general population), PLIN5: 13041A (major allele in general population)>G (minor allele in general population), and PLIN6: 14995A (major allele in general population)>T (minor allele in general population). For example, at PLIN4 locus, an individual may be a homozygote GG, a heterozygote GA or a homozygote AA.

One particularly useful locus in the method according to the present invention is the PLIN4 locus or any other locus in very tight linkage disequilibrium with the PLIN4 locus. As used herein, a “very tight linkage disequilibrium” means a polymorphic marker that co-segregates 100% with the allele “A” in the PLIN4 locus. Therefore, any tightly linked polymorphic marker discovered by in-silico searches or by resequencing of carriers of the PLIN4 locus could be also used as diagnostic tools.

Biological sample used as a source material for isolating the nucleic acids in the instant invention include, but are not limited to solid materials (e.g., tissue, cell pellets, biopsies, hair follicle samples, buccal smear or swab) and biological fluids (e.g. blood, saliva, amniotic fluid, mouth wash, urine). Any biological sample from a human individual comprising even one cell comprising nucleic acid, can be used in the methods of the present invention. Nucleic acid molecules of the instant invention include DNA and RNA, preferably genomic DNA, and can be isolated from a particular biological sample using any of a number of procedures, which are well-known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russel, Cold Spring Harbor Laboratory Press, 2001.

The PLIN polymorphisms of the present invention can be detected from the isolated nucleic acids using techniques including direct analysis of isolated nucleic acids such as Southern Blot Hybridization (DNA) or direct nucleic acid sequencing (Molecular Cloning: A Laboratory Manual, 3rd Ed., Sambrook and Russel, Cold Spring Harbor Laboratory Press, 2001).

An alternative method useful according to the present invention for direct analysis of the PLIN polymorphisms is the INVADER® assay (Third Wave Technologies, Inc (Madison, Wis.). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535).

Preferably, a PCR based techniques are used. After PCR, the polymorphic nucleic acids can be identified using, for example direct sequencing with radioactively or fluorescently labeled primers; single-stand conformation polymorphism analysis (SSCP), denaturating gradient gel electrophoresis (DGGE); and chemical cleavage analysis, all of which are explained in detail, for example, in the Molecular Cloning: A Laboratory Manual, 3rd Ed., Sambrook and Russel, Cold Spring Harbor Laboratory Press, 2001.

The polymorphisms are preferably analyzed using methods amenable for automation such as the different methods for primer extension analysis. Primer extension analysis can be preformed using any method known to one skilled in the art including PYROSEQUENCING™ (Uppsala, Sweden); Mass Spectrometry including MALDI-TOF, or Matrix Assisted Laser Desorption Ionization—Time of Flight; genomic nucleic acid arrays (Shalon et al., Genome Research 6(7):639-45, 1996; Bernard et al., Nucleic Acids Research 24(8):1435-42, 1996); solid-phase mini-sequencing technique (U.S. Pat. No. 6,013,431, Suomalainen et al. Mol. Biotechnol. June; 15(2):123-31, 2000); ion-pair high-performance liquid chromatography (Doris et al. J. Chromatogr. A May 8; 806(1):47-60, 1998); and 5′ nuclease assay or real-time RT-PCR (Holland et al. Proc Natl Acad Sci USA 88: 7276-7280, 1991), or primer extension methods described in the U.S. Pat. No. 6,355,433. Nucleic acids sequencing, for example using any automated sequencing system and either labeled primers or labeled terminator dideoxynucleotides can also be used to detect the polymorphisms. Systems for automated sequence analysis include, for example, Hitachi FMBIO® and Hitachi FMBIO® II Fluorescent Scanners (Hitachi Genetic Systems, Alameda, Calif.); Spectrumedix® SCE 9610 Fully Automated 96-Capillary Electrophoresis Genetic Analysis System (SpectruMedix LLC, State College, Pa.); ABI PRISM® 377 DNA Sequencer; ABI® 373 DNA Sequencer; ABI PRISM® 310 Genetic Analyzer; ABI PRISM® 3100 Genetic Analyzer; ABI PRISM® 3700 DNA Analyzer (Applied Biosystems, Headquarters, Foster City, Calif.); Molecular Dynamics Fluorlmager™ 575 and SI Fluorescent Scanners and Molecular Dynamics Fluorlmagemm 595 Fluorescent Scanners (Amersham Biosciences UK Limited, Little Chalfont, Buckinghamshire, England); GenomyxSC™ DNA Sequencing System (Genomyx Corporation (Foster City, Calif.); Pharmacia ALF™ DNA Sequencer and Pharmacia ALFexpress™ (Amersham Biosciences UK Limited, Little Chalfont, Buckinghamshire, England).

PCR, nucleic acid sequencing and primer extension reactions for one nucleic acid sample can be performed in the same or separate reactions using the primers designed to amplify and detect the polymorphic PLIN nucleotides.

In one embodiment, the invention provides a nucleic acid chip including the polymorphic PLIN1, PLIN4, PLIN5, and PLIN6 alleles for the screening of individual with a risk of PLIN-associated obesity and/or obesity-related diseases, inclusing cardiovascular disease, or PLIN-associated protection from obesity and/or obesity-related diseases, such as cardiovascular disease. Such chip can include any number of other obesity-associated mutations and polymorphisms including but not limited to leptin, leptin receptor, MC4R and others. A list of obesity associated genes and polymorphisms can be found, for example, in Chagnon, Y. C., Perusse, L., Weisnagel, S. J., Rankinen, T. and Bouchard, C. The Human Obesity Gene Map: The 1999 Update. Obesity Research 8 (1): 89-117, 2000, and on the web at http://www.obesity.chair.ulaval.ca/genemap.html.

In one embodiment, the invention provides a kit comprising one or more primer pairs capable of amplifying the PLIN nucleic acid regions comprising the obesity associated polymorphic nucleotides of the present invention; buffer and nucleotide mix for the PCR reaction; appropriate enzymes for PCR reaction in same or separate containers as well as an instruction manual defining the PCR conditions, for example, as described in the Example below, as well as listing the obesity associated alleles and haplotypes as described in this specification. The kit may further comprise nucleic acid probes, preferably those listed on Table 1, either in dry form in a tube or a vial or in a buffer. In the preferred embodiment, these primers are the ones listed on Table 1. Primers may also be provided in the kit in either dry form in a tube or a vial, or alternatively dissolved into an appropriate aqueous buffer. The kit may also comprise primers for the primer extension method for detection of the specific PLIN polymorphisms as described above.

In one embodiment, the components of the kit are part of a kit providing for multiple obesity associated genes, polymorphisms and mutations known in to one skilled in the art.

The detection of at least one allele “A” at PLIN4 locus is indicative of the carrier individual being relatively resistant to weight regulation using diet, such as low-calorie diet.

The low-energy, low calorie, or calorie-restricted diet, as used herein, refers to a standard hypocaloric diet with an energy content being approximately 1200 kcal/day (lipids: 52 g, proteins: 62 g, and carbohydrates: 121 g). Usually, standard low calorie diets are considered those in the range of 1,000-1,500 kilocalories per day. Other low calorie diets require clinical supervision and are known as very low calorie diets (400-500 kilocalories per day), but are also encompassed in the term “low calorie diet” as used in this specification.

The overweight individual, as used herein, refers to an individual fulfilling the normal definition of overweight individual as defined by the medical knowledge at the time of diagnosis. Useful criteria for defining an individual as overweight, include, but are not limited to the following criteria: body mass index (BMI) of 25-29.9, male individual with a waist measurement greater than 40 inches (102 cm), female individual with a waist measurement greater than 35 inches (88 cm), and all individuals with a waist-to-hip ratio of 1.0 or higher.

The obese individual as used herein refers to an individuals fulfilling the normal definition of overweight individual as defined by the medical knowledge at the time of diagnosis. Useful criterium for defining an individual as obese, include, but is not limited to body mass index (BMI) of 30 or over.

An adult individual within normal weight range as used herein, refers to individuals defined as normal weight at the time of observation. For example, an individual with BMI of about 18.5-24.9, would currently be considered as normal weight, and an individual of BMI under 18.5, would be considered as underweight (Centers for Disease Control and Prevention BMI Information at http://www.cdc.gov/nccdphp/dnpa/bmi/bmi-adult.htm).

The normal, over and under weight definitions vary in children and teenagers. Information at the CDC web-site http://www.cdc.gov/nccdphp/dnpalbmi/bmi-for-age.htm can be used as reference material in evaluating normal weight in children.

However, as the knowledge of the effects of weight on health increases, these definitions may change. Thus, the term normal weight, under- and overweight, and obese, as used herein, include the definitions at the time of observation of the individual in light of then current medical knowledge.

In one embodiment, the invention provides a kit for determining susceptibility to weight-loss using dietary intervention, including a low-calorie diet. Such kit includes instructions that if an allele “A” at PLIN4 locus is detected in the tested individual, the individual is unlikely responsive to low-calorie diet as a weight-reduction means, and that if no allele “A” at PLIN4 locus is detected, the individual is susceptible for weight-loss using dietary intervention, such as low-calorie diet. The kit also includes means to detect polymorphisms in the PLIN loci, preferably at least PLIN4 locus. The kit may also include only a detection means for detecting the A allele at PLIN4 locus, wherein a negative result, for example, no PCR product, or no signal, is indicative of the individual being susceptible for weight-loss management using dietary means. Because both heterozygotes (A/G at PLIN4 locus) and homozygotes (A/A at PLIN4 locus) are resistant to dietary weight-loss regimes, a kit must be able to detect at least the allele A or any allele in very tight linkage disequilibrium with allele A of the PLIN4 locus.

Similar kit can also be used to determine whether any individual is resistant to weight change. Thus the kit could be used in combination with weight management programs equally well in normal weight, underweight and overweight individuals.

### EXAMPLES

A recent study examining perilipin expression in humans, has found that perilipin was elevated in obese subjects (18). Moreover, in the first large population-based study we has demonstrated that variations at the perilipin (PLIN) locus are associated with obesity risk (19), finding that was subsequently supported by other studies in white and Asian populations (Qi L, et al., Intragenic linkage disequilibrium structure of the human perilipin gene (PLIN) and haplotype association with increased obesity risk in a multiethnic Asian population, J Mol Med. 2005 Mar. 16; and Qi L, et al., Gender-specific association of a perilipin gene haplotype with obesity risk in a white population. Obes Res. 2004 November; 12 (11):1758-65). These results prompted us to perform the current study aimed to analyze the influence of PLIN polymorphisms on anthropometrical measures in massively obese subjects as well as to examine the potential gene-diet interaction between PLIN polymorphisms and an energy-restricted diet on the ability to lose weight during a 1-year follow-up intervention.

Subjects and Methods

Patients and study design: The present study included 150 obese patients (29 men and 121 women aged 18-68 years) referred to the Endocrinology Unit of the University General Hospital in Valencia, Spain for diagnostic and weight reduction treatments related to obesity. These patients were randomly selected among those obese subjects referred consecutively from May 2001 to September 2002, and who had normal thyroid function and no concomitant renal, hepatic, cardiac disease or Cushing disease. Pregnant or nursing women were also excluded. All patients were Caucasian and the mean age was 48±14 years. Body mass index (BMI) ranged from 30 to 79 Kg/m2, with 88% of patients having a BMI≧35 Kg/m2. All participants provided informed consent and the study protocol was approved by the Ethics Committees of the Valencia University and the University General Hospital.

At baseline, anthropometric, biochemical, and clinical characteristics were determined in all patients. In addition, genomic DNA was isolated from blood and stored for further genetic analysis. Weight reduction treatments including diet, drugs or surgery were recommended to each obese patient according to standard clinical guidelines (20). Bariatric surgery (21) was recommended to 13 patients. 42 patients received weight-loss medications (orlistat, sibutramine, antidepresants, or fiber) combined with diet, and 92 patients were prescribed to receive an energy-restricted diet. Patients assigned to the energy restricted diet with no medication for weight loss (61% of patient at baseline) were invited to participate in the one-year follow-up study to investigate if PLIN polymorphism modulate the weight loss in response to diet. As PLIN genotypes in these patients were determined at the end of the follow-up, the design of this study can be classified as a double-blinded paralleled randomized trial because no one had previous information about the group assignment. The randomization of individuals is provided by Mendelian randomization, the term applied to the random assortment of alleles at the time of gamete formation (22).

Dietary intervention: Forty-eight motivated patients (9 men and 39 women) completed the one-year dietary follow-up treatment and had complete dataset at each time point. All patients started with a 2-weeks very-low energy diet (Modifast; NOVARTIS Nutrition, Bern, Switzerland) providing 603 Kcal/day (lipids: 13.5 g, proteins: 52.5 g and carbohydrates: 67.5 g) under highly controlled hospital conditions. Thereafter, conventional food was introduced and patients were advised by a dietician to consume a standard hypocaloric diet with an energy content being approximately 1200 kcal/day (lipids: 52 g, proteins: 62 g, and carbohydrates: 121 g) for one year. The patients were given dietary instructions based on an education system consisting of isoenergetic interchangeable units. Three follow-up evaluations were performed at 3, 6 and 12 months. All evaluations were conducted at the Endocrinology Unit of the University General Hospital. Adherence to diet was confirmed in these evaluations. None of the obese patients was involved in an exercise program.

Measurements: Anthropometrical measurements were taken using standard techniques (19): weight with light clothing by digital scales; height without shoes by fixed stadiometer. Waist circumference was measured midway between the lower rib margin and the iliac crest in the horizontal plane. Hip circumference was measured at the point yielding the maximum circumference over the buttocks. In the follow-up study, the subjects were weighed when they visited the endocrinology unit at baseline, 3, 6 and 12 months of the study. All measurements were done on the same equipment by the same personal each time.

Venous blood was collected into EDTA-containing glass tubes. Plasma total cholesterol, fasting TAGs, high-density lipoprotein cholesterol (HDL-C), low-density lipoprotein-cholesterol and fasting glucose were measured at baseline as previously described (19). A baseline questionnaire was used to obtain demographic information, education, health status, menopausal status, medication, tobacco smoking, alcohol consumption, physical activity and weight history for the prior years. Education was classified into three categories: primary, secondary (high-school) and university. Current smokers were defined as those smoking at least one cigarette per day. Subjects with any amount of alcohol consumed were classified as drinkers. Physical activity was estimated from questions about regular leisure-time physical sports, and subjects were categorized as sedentary (no physical exercise), or active (23). Subjects were classified as having type 2 diabetes if they were on hypoglycemic drug therapy for diagnosed type 2 diabetes, of if they had fasting plasma glucose levels >126 mg/dL (24).

PLIN genotyping: Four polymorphisms at the PLIN locus (PLIN1: 6209T>C, PLIN4: 11482G>A, PLIN5: 13041A>G, and PLIN6: 14995A>T) were genotyped. Genotyping was carried out using Single Nucleotide Extension as previously reported (19) using the ABI Prism SnaPshot multiplex system on an ABI Prism 3100 genetic analyzer (Applied Biosystems, Foster City, Calif.). Standard good laboratory practices were undertaken to assure the accuracy of genotype data.

Statistical analysis: χ² tests were used to test differences between observed and expected frequencies, assuming Hardy-Weinberg equilibrium, to test linkage disequilibrium, and to test differences in percentages of alleles. Pairwise linkage disequilibrium coefficients were estimated by the LINKAGE program. D and D′ (D/Dmax) coefficients were calculated. Normal distribution for all continuous variables was tested and triglycerides were logarithmically transformed. Carriers of the less common allele were grouped and compared with wild-type, i.e., the more common allele carrying homozygotes. At baseline, Student's t test for independent groups were applied to compare crude means between genotypes. In addition, to estimate and to compared adjusted means, analysis of covariance was used to test the null hypotheses of no association between genetic variants and obesity-related phenotypes. The main covariates were gender, age, tobacco smoking, alcohol consumption, physical activity, type 2 diabetes, education and menopausal status in women. Homogeneity of allelic effects according to gender or to other factors was tested by introducing the corresponding terms of interaction in the more parsimonious multivariate model. Standard regression diagnostic procedures Were used to ensure the appropriateness of these models. In the dietary intervention follow-up study analysis of covariance for repeated measures (at baseline, 3, 6 and 12 months) was used to test the gene-diet interaction in determining weight loss as well as to control for the potential confounders. The Statistical Package for Social Sciences (SPSS, v.11.5) was used for statistical analyses.

Results

Table 1 shows demographic, anthropometric, clinical, biochemical, and lifestyle characteristics of the 150 obese patients (29 men and 121 women) by gender at baseline. Subjects had a high degree of obesity (mean BMI, 42±8 kg/m2), a low educational level and were sedentary (more than 80%). Sixty two women (51%) were postmenopausal. PLIN genotypes were determined in these patients. As differences by gender in the genotype distributions were not significant for any polymorphism, data for men and women were analyzed together (Table 2). Genotype distributions did not deviate from Hardy-Weinberg expectations for any SNP. A strong pairwise linkage disequilibrium was observed between the PLIN1 (6209T>C) and the PLIN4 (11482G>A) polymorphisms (D′: 0.96; p<0.001). Much lower, but still statistically significant, positive linkage disequilibrium was observed between the other polymorphisms: PLIN6 (14995A>T) and PLIN5 (11482G>A), with D′ coefficients of 0.46 and 0.15, respectively).

At baseline, we studied the association between these SNPs and anthropometric variables in the 150 obese patients. To increase the statistical power and after having verified the presence of an allelic effect compatible with a dominant, or at least, a co-dominant model, carriers of the less common allele were grouped and compared with wild-type subjects. We did not find significant gene-gender interactions when evaluated homogeneity by gender, and data for men and women were analyzed together (results are presented gender-adjusted). The PLIN 4 (11482G>A) polymorphism was the only one that was statistically associated with weight and BMI in the obese patients. Table 3 shows anthropometric, biochemical and clinical characteristics of the study subjects according to the PLIN 4 (11482G>A) polymorphism. At baseline, we observed that carriers of the A allele at the PLIN 4 (11482G>A) polymorphism had significantly less body weight (−7.7%) and BMI than GG homozygotes. This association remained statistically significant even after additional control for potential confounders (smoking, drinking, physical activity, education, type 2 diabetes and menopausal status in women). Due to the high linkage disequilibrium between the PLIN 4 (11482G>A) and PLIN 1 (6209T>C) polymorphisms, lower mean body weight was also observed in carriers of the 6209 C allele at the PLIN1 locus; however the difference did not reach the statistical significance (P=0.071).

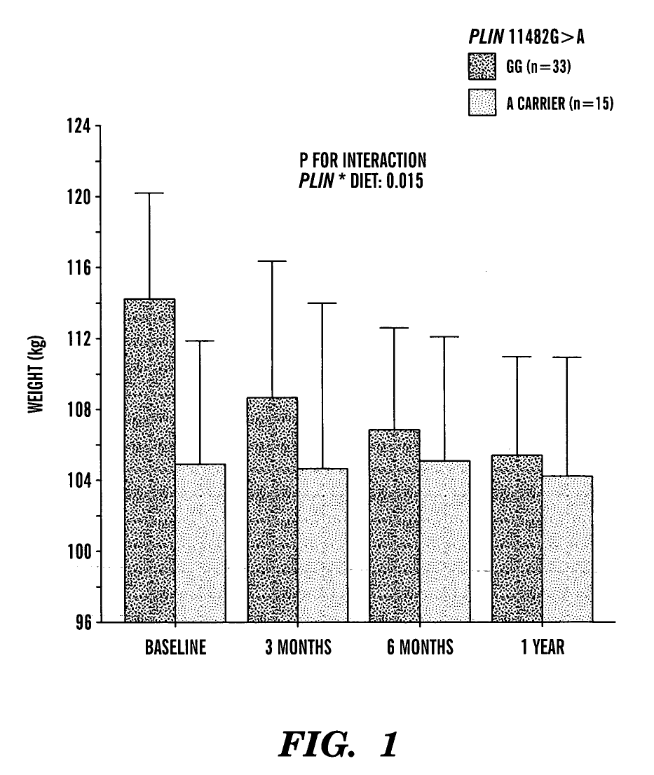

Following the collection of baseline data, weight reduction treatments were prescribed. Bariatric surgery was recommended to 13 patients. 42 patients received weight-loss medications (orlistat, sibutramine, antidepresants, or fiber) combined with diet, and 92 patients were prescribed to receive only an energy-restricted diet. Patients assigned to the energy-restricted diet, and who were not receiving medication for weight loss, were invited to participate in the one-year follow-up study. Forty-eight patients (9 men and 39 women) were followed for the entire 1-year diet period and had complete dataset at each time point (at 3, 6 and 12 months). We found a statistically significant (P=0.015) gene-diet interaction between the PLIN4 (11482G>A) polymorphism and body weight decrease in response to diet. No statistically significant interaction terms were found for the other PLIN polymorphisms, although the PLIN1 (6209T>C) paralleled the effect of the PLIN4 SNP due to the high degree of linkage disequilibrium. Table 3 shows baseline characteristics of the 48 patients that completed the energy-restricted follow-up study according to the PLIN4 (11482G>A) polymorphism. No statistically significant differences were observed in baseline variables between the two genotype groups. Smoking, drinking, education, physical activity, diabetes and menopausal status in women did not differ either. Interestingly, although both genotype groups received the same energy-restricted diet, weight loss differed different significantly between the two groups. FIG. 1 shows means of body weight (gender and age adjusted) in the 48 obese subjects who completed the study for weight reduction under the energy-restricted diet according to the PLIN4 (11482G>A) polymorphism. The intervention resulted in a significant decrease in mean body weight (from 114.3±3.9 kg at baseline to 105.5±3.5/kg at 1-year; P-lineal trend: 0.020) in patients with wild-type genotype (GG).

Conversely, patients with the variant allele (A) did not show significant changes in mean body weight (from 105.0±4.6 kg at baseline to 104.3±4.4 kg at 1-year; P-lineal trend: 0.985). The difficulty in losing weight among A allele-carriers was consistently observed at 3, 6 and 12 months, reducing the likelihood that this finding was observed by chance. Further adjustment of the model for education, smoking, physical activity, diabetes and menopausal status did not modified the statistical significance of the gene-diet interaction. Furthermore, the potential interaction effect with diabetes status was also tested and we did not observe a statistically significant term (P=0.902). Table 4 shows adjusted means of body weight in at baseline and after 3, 6 and 12 months of low energy diet in non-diabetic and diabetic subjects depending on the PLIN4 (11482G>A) polymorphism. In both non-diabetic and diabetic subjects, body weight decreased over the course of the intervention in subjects homozygotes for the PLIN 11482G allele. However, in carriers of the variant allele (A) there were no relevant changes. This modification of the effect was statistically significant (P=0.041) in non-diabetic subjects. In diabetic subjects, although the magnitude of the effect was similar, due to the lower sample size the interaction term did not reach the statistical significance.

Discussion

In the present study we have confirmed the role of the PLIN4 (11482G>A) polymorphisms in determining body-weight in humans. In a previous investigation, carried out in subjects randomly selected from the general population in the same geographical area (19), we have reported that PLIN4 (11482G>A) polymorphism was significantly associated with body weight and obesity risk. Thus, the 11482A variant-allele was associated with a lower obesity risk (Odds ratio (OR)=0.56, 95% CI: 0.36-0.89) and with a mean decrease of −2.2 Kg (about a 3.5%) of body weight of body weight) in women. At baseline, in this massively obese population, the 11482A variant allele was associated with a three times higher decrease in body weight (about 9%) than in the general population, suggesting a more prominent role of variations in the PLIN locus in morbidly obese subjects. The maximization of genetic effects of important candidate gene for obesity in severely obese subjects has been pointed out by Bell et al (26) in their recent review about the genetics of human obesity. Another interesting finding in the comparison between morbidly obese patients and the general population is that the association between PLIN polymorphisms and body weight was not observed in men from the general population (19). In obese patients, the PLIN 11482A variant allele was associated with lower body-weight in both men and women. The reason for this discrepancy is unknown, however it is likely that the higher adiposity observed in severely obese men as compared with men from the general population largely contribute to this association. Although in another large population study analyzing the association between PLIN polymorphisms and obesity-related variables in white American subjects, we have also found a gene-gender interaction because no association were found in men (26), results from animal studies reported similar effects in both male and female mice (17). Therefore, more human studies analyzing PLIN variation in men are needed to confirm if the effect of PLIN variation on anthropometric variables in men depends on the obesity degree.

In agreement with results from animal models (16, 17), the “protective” effects of the PLIN4 (11482G>A) polymorphism observed in obese patients at baseline, as well as in women from the general population, are compatible with a reduced expression of the 11482A-variant allele as compared with GG homozygotes. Data from animal models have consistently shown that targeted disruption of the perilipin gene results in healthy mice that are much leaner and more muscular than wild-type controls and had increased levels of basal lipolysis (16, 17). In support to this hypothesis, Kern et al (18) in a study carried out in 44 healthy subjects (5 men and 39 women), demonstrated a significant positive relationship between perilipin expression and obesity. Although the PLIN4 (11482G>A) polymorphism is located in an intron, and it do not appears to be traditionally functional, Mottagui-Tabar et al (13) in a study carried out in human fat cells of obese women, have demonstrated that the perilipin protein content was markedly decreased and lypolisis increased in carriers of the 11482A-variant allele supporting the observed results.

Despite the apparent “protective” role of the 11482A-variant allele associated with lower body-weight at baseline, our dietary intervention follow-up study has revealed that carriers of this allele at the PLIN locus are more resistant to weight loss in response to an energy-restricted diet than GG homozygotes. So, patients with the 11482A variant-allele did not show significant changes in mean body weight (−0.7 Kg from baseline to 1-year). Conversely, subjects homozygotes for the 11482G allele had a greater and statistically significant mean weight loss (−9 Kg from baseline to 1-year) during the same 1-year low energy diet regimen. This is the first study on weight loss and PLIN variation in humans in response to a long-term energy-restricted diet and the molecular mechanism to explain the observed results remains to be explained. However, considering the results obtained in perilipin knockout mouse that are resistant to diet induced obesity (17), we can speculate that carriers of the 11482A-variant allele (associated with less perilipin expression) experiment a “buffer” effect by which body-weight regulation in this subjects is more independent of the energy intake than in GG homozygotes. Several pathways might be involved in this “buffer” effect including the leptin signaling (16, 17, 18) and even the general modulation by transcription factors such as PPARs (27). Accordingly, Castro-Chavez et al (28) analyzed the gene-expression profile of white adipose tissue of plin(−/−) and plin(+/+) mice, showing that the disruption of perilipin leads to extensive changes in gene expression in the adipose tissue compatible with the implication of a set of transcriptional factors or co-activators as mediators for observed changes.

Another aspect that remains to be investigated is macronutrient influence in the PLIN-diet interaction. In our study, the dietary target for fat content in the low energy diet was 39% of energy (21%, proteins and 40%, carbohydrates). This relatively high fat content reflects the habitual dietary fat intake in Spain that is characterized by a typical Mediterranean diet in which olive oil is the main fat consumed (29). Little is known about the ways in which macronutrients and energy restriction affect the regulation of adipose tissue gene expression, and if a very-low fat diet may have the same effect in modulating weight loss in carriers of the PLIN 11482A-variant allele. On this regard, Viguerie et al (30) in the NUGENOB project, carried out an study in two groups of 25 obese subjects following 10-week hypocaloric diet programmes with either 20-25 (low-fat) or 40-45% (hig.fat) of total energy derived from fat to investigate if gene expression in adipose tissue is dependent on the energy restriction as such or on the macronutrient composition of the diet. They found that ten genes were regulated by energy restriction; however, none of the genes showed a significantly different response to the diets concluding that energy restriction and/or weight loss rather than the ratio of fat: carbohydrate in a low-energy diet is of importance in modifying the expression of genes in the human adipose tissue.

In conclusion, our results show that the 11482G>A polymorphism predicts outcome of body-weight reduction strategies based on low-calorie diets. Carriers of the A allele have higher stability in the mechanisms that control the energy balance and body-weight. This “buffer” effect could also explain a higher resistance in carriers of this allele to increase body-weight in response to a high-fat diet. Because our study is the first longitudinal study investigating the association between the PLIN polymorphisms and weight loss in response to diet, there is a need to confirm the present findings in other interventional studies. However, our study has important strengths that add evidence to obtained results: The difficulty in losing weight among carriers of the variant allele was consistently observed at 3, 6 and 12 months, reducing the likelihood that this finding was observed by chance; the fully-blinded nature of this study (neither the patient, the physician nor the technical staff did know the PLIN genotype of the patient) prevented us of the potential bias of some potential intervention differences between the groups of GG homozygotes and A allele-carriers; and the Mendelian randomization (22) provides a random distribution of individuals in the two genotype groups comparable to a randomized trial.

The references cited herein and throughout the specification are herein incorporated by reference in their entirety.

### REFERENCES

1. Rossner S. Obesity: the disease of the twenty-first century. Int J Obes Relat Metab Disord. 2002 December; 26 Suppl 4:S2-4. Review.
2. Roth J, Qiang X, Marban S L, Redelt H, Lowell B C. The obesity pandemic: where have we been and where are we going? Obes Res. 2004 November; 12 Suppl 2:88S-101S.
3. Speakman J R. Obesity: the integrated roles of environment and genetics. J Nutr. 2004 August; 134(8 Suppl):2090S-2105S. Review.
4. Klein S. Clinical trial experience with fat-restricted vs. carbohydrate-restricted weight-loss diets. Obes Res. 2004 November; 12 Suppl 2:141S-4S.
5. McAuley K A, Hopkins C M, Smith K J, McLay R T, Williams S M, Taylor R W, Mann J I. Comparison of high-fat and high-protein diets with a high-carbohydrate diet in insulin-resistant obese women. Diabetologia. 2005 January; 48(1):8-16. Epub 2004 Dec. 23.
6. Dansinger M L, Gleason J A, Griffith J L, Selker H P, Schaefer E J. Comparison of the Atkins, Omish, Weight Watchers, and Zone diets for weight loss and heart disease risk reduction: a randomized trial. JAMA. 2005; 293:43-53.
7. Ordovas J M, Corella D. Nutritional genomics. Annu Rev Genomics Hum Genet. 2004; 5:71-118. Review
8. Tremblay A, Perusse L, Bouchard C. Energy balance and body-weight stability: impact of gene-environment interactions. Br J Nutr. 2004 August; 92 Suppl 1:S63-6. Review.
9. Greenberg A S, Egan J J, Wek S A, Moos M C Jr, Londos C, Kimmel A R. Isolation of cDNAs for perilipins A and B: sequence and expression of lipid droplet-associated proteins of adipocytes. Proc Natl Acad Sci USA. 1993 Dec. 15;90(24):12035-9.
10. Blanchette-Mackie E J, Dwyer N K, Barber T, Coxey R A, Takeda T, Rondinone C M, Theodorakis J L, Greenberg A S, Londos C. Perilipin is located on the surface layer of intracellular lipid droplets in adipocytes. J Lipid Res. 1995 June; 36(6):1211-26.
11. Brasaemle D L, Rubin B, Harten I A, Gruia-Gray J, Kimmel A R, Londos C. Perilipin A increases triacylglycerol storage by decreasing the rate of triacylglycerol hydrolysis. J Biol Chem. 2000 Dec. 8; 275(49):38486-93.
12. Souza S C, de Vargas L M, Yamamoto M T, Lien P, Franciosa M D, Moss L G, Greenberg A S. Overexpression of perilipin A and B blocks the ability of tumor necrosis factor alpha to increase lipolysis in 3T3-L1 adipocytes. J Biol Chem. 1998 Sep. 18; 273(38):24665-9.
13. Mottagui-Tabar S, Ryden M, Lofgren P, Faulds G, Hoffstedt J, Brookes A J, Andersson I, Arner P. Evidence for an important role of perilipin in the regulation of adipocyte lipolysis. Diabetologia 2003; 46:789-97.
14. Sztalryd C, Xu G, Dorward H, Tansey J T, Contreras J A, Kimmel A R, Londos C. Perilipin A is essential for the translocation of hormone-sensitive lipase during lipolytic activation. J Cell Biol. 2003 Jun. 23; 161(6):1093-103.
15. Fricke K, Heitland A, Maronde E. Cooperative activation of lipolysis by protein kinase A and protein kinase C pathways in 3T3-L1 adipocytes. Endocrinology. 2004 November; 145(11):4940-7. Epub 2004 Jul. 29.
16. Martinez-Botas J, Anderson J B, Tessier D, Lapillonne A, Chang B H, Quast M J, Gorenstein D, Chen K H, Chan L. Absence of perilipin results in leanness and reverses obesity in Leprdb/db mice. Nature Genetics 2000; 26:474-79.
17. Tansey J T, Sztalryd C, Gruia-Gray J, Roush D L, Zee J V, Gavrilova O, Reitman M L, Deng C X, Li C, Kimmel A R, Londos C. Perilipin ablation results in a lean mouse with aberrant adipocyte lipolysis, enhanced leptin production, and resistance to diet-induced obesity. Proc. Natl. Acad. Sci., USA 2001; 98:6494-99.
18. Kern P A, Di Gregorio G, Lu T, Rassouli N, Ranganathan G. Perilipin expression in human adipose tissue is elevated with obesity. J Clin Endocrinol Metab. 2004; 89:1352-8.
19. Qi L, Corella D, Sorli J V, Portoles O, Shen H, Coltell O, Godoy D, Greenberg A S, Ordovas J M. Genetic variation at the Perilipin (PLIN) locus is associated with obesity-related phenotypes in White women. Clin Genet 2004; 66:299-310.
20. Aronne L J. Obesity as a disease: etiology, treatment, and management considerations for the obese patient. Obes Res. 2002 December; 10 Suppl 2:95S-96S.
21. Buchwald H, Avidor Y, Braunwald E, Jensen M D, Pories W, Fahrbach K, Schoelles K. Bariatric surgery: a systematic review and meta-analysis. JAMA. 2004 Oct. 13; 292(14):1724-37. Review.
22. Smith G D, Ebrahim S. Mendelian randomization: prospects, potentials, and limitations. Int J Epidemiol. 2004 February; 33(1):30-42.
23. Corella D, Guillen M, Saiz C, Portoles O, Sabater A, Cortina S, Folch J, Gonzalez J I, Ordovas J M. Environmental factors modulate the effect of the APOE genetic polymorphism on plasma lipid concentrations: ecogenetic studies in a Mediterranean Spanish population. Metabolism. 2001 August; 50(8):936-44.
24. Expert Committee on the Diagnosis and Classification of Diabetes Mellitus. Report of the Expert Committee on the Diagnosis and Classification of Diabetes Mellitus. Diabetes Care 1997; 20:1183-97.
25. Bell C G, Walley A J, Froguel P. The genetics of human obesity. Nat Rev Genet. 2005; 6: 221-230.
26. Qi L, Shen H, Larson I, Schaefer E J, Greenberg A S, Tregouet D A, Corella D, Ordovas J M. Gender-specific association of a perilipin gene haplotype with obesity risk in a white population. Obes Res. 2004 November; 12(11):1758-65.
27. Arimura N, Horiba T, Imagawa M, Shimizu M, Sato R. The peroxisome proliferator-activated receptor gamma regulates expression of the perilipin gene in adipocytes. J Biol Chem. 2004 Mar. 12; 279(11):10070-6.
28. Castro-Chavez F, Yechoor V K, Saha P K, Martinez-Botas J, Wooten E C, Sharma S, O'Connell P, Taegtmeyer H, Chan L. Coordinated upregulation of oxidative pathways and downregulation of lipid biosynthesis underlie obesity resistance in perilipin knockout mice: a microarray gene expression profile. Diabetes. 2003 November; 52(11):2666-74.
29. Serra-Majem L, de la Cruz J N, Ribas L, Salleras L. Mediterranean diet and health: is all the secret in olive oil? Pathophysiol Haemost Thromb. 2003 September-2004 December; 33(5-6):461-5.
30. Viguerie N, Vidal H, Arner P, Holst C, Verdich C, Avizou S, Astrup A, Saris W H, Macdonald I A, Klimcakova E, Clement K, Martinez A, Hoffstedt J, Sorensen T I, Langin D; Nutrient-Gene Interactions in Human Obesity—Implications for Dietary Guideline (NUGENOB) project. Adipose tissue gene expression in obese subjects during low-fat and high-fat hypocaloric diets. Diabetologia. 2005 January; 48(1):123-31.

We claim:

1. A method for predicting a likelihood of success of an individual in a weight management program, the method comprising obtaining a biological sample comprising nucleic acid of an individual, genotyping the nucleic acid for polymorphic markers at a perilipin locus, wherein the presence of either one or two PLIN4 “A” alleles is indicative of decreased likelihood of success of the individual to a weight management program comprising a component of dietary intervention and wherein the absence of PLIN4 “A” allele is indicative of increased likelihood of success of the individual in a weight management program.

2. A method of determining whether an individual is a suitable candidate for weight management program comprising a component of dietary intervention, the method comprising genotyping the PLIN locus in a nucleic acid sample of the over-weight or obese individual, wherein the absence of PLIN4 allele “A” is indicative of the individual being a suitable candidate for weight management program comprising a component of dietary intervention.

3. A method of determining whether an individual is not a suitable candidate for weight-management program comprising dietary intervention alone, the method comprising genotyping at least one polymorphic marker in the perilipin (PLIN) locus of the overweight or obese individual, wherein the presence of one or two PLIN4 allele “A” is indicative of the individual not being a suitable candidate for weight-management program comprising dietary intervention alone.

4. The method of claims 1, 2, or 3, wherein the individual is overweight or obese.

5. The method of claim 2, 3, or 4, wherein the dietary intervention comprises a low energy diet.

6. A kit for determining, whether an individual is or is not an appropriate candidate to weight management program, the kit comprising one or more primer pairs for genotyping at least one polymorphic marker at the perilipin (PLIN) locus, wherein the PLIN4 polymorphic locus, and an instruction leaflet that explains that detection of at least one allele “A” at PLIN4 locus in indicative of the individual as being not susceptible for weight management program, and that detection of no PLIN4 allele “A” is indicative of that individual being susceptible to weight management program.

7. The kit of claim 6, wherein the weight management program comprises a component of dietary intervention.

8. The kit of claim 7, wherein the dietary intervention comprises a low calorie diet.
